(19) United States
(12) Patent Application Publication
(10) Pub. No.: US 2004/0230126 A1
(43) Pub. Date: Nov. 18, 2004

(54) ECG monitoring apparatus with incorporated printer

(75) Inventors: Mark E. Pingel, Camillus, NY (US); Peter W. Galen, Portland, OR (US); Peter W. Glocker, Skaneateles, NY (US); Todd B. Abernethy, Skaneateles, NY (US); Scott W. Osiecki, Skaneateles, NY (US)

Correspondence Address: WALL MARJAMA & BILINSKI, 101 SOUTH SALINA STREET, SUITE 400, SYRACUSE, NY, 13202, US

(73) Assignee: Welch Allyn, Inc.

(21) Appl. No.: 10/728,208

(22) Filed: Dec. 4, 2003

Related U.S. Application Data
(60) Provisional application No. 60/431,459, filed on Dec. 5, 2002.

(51) Int. Cl. A61B 5/0402

(52) U.S. Cl. 600/509

(57) ABSTRACT

An ECG monitoring apparatus is coupled to a conventional (e.g., plain paper) printer. The apparatus can be mechanically coupled as a removable portion of the assembly or can be integrated directly into the printer design.

### CROSS REFERENCE TO RELATED APPLICATIONS

This application is based upon provisional patent application, U.S. Ser. No. 60/431,459, filed Dec. 5, 2002, under 35 USC §119(e) the entire contents of which are herein incorporated by reference.

### FIELD OF THE INVENTION

The invention relates to the field of electrocardiography (ECG), and more particularly to apparatus and a related method for adapting ECG monitoring equipment with conventional (e.g., plain paper) printers.

### BACKGROUND OF THE INVENTION

It is commonly known that ECGs are used to monitor electrical activity of the heart of a patient. A set of electrodes and leadwires are attached to the body of a patient and the resulting waveforms detected by each of the leads are outputted, typically to a thermal paper printer attached to the ECG monitor. Thermal paper is quite expensive and often difficult to work with in that this paper easily smears, such that the output is often not very distinguishable.

PC-based ECG monitoring systems have been introduced, such as those developed by Marquette and Hewlett Packard, among others, permit results to be printed from a computer, but to date there is no known technique for interconnecting ECG monitoring apparatus with conventional printers without providing computer interconnections.

### SUMMARY OF THE INVENTION

It is a primary object of the present invention to solve the above-noted problems of the prior art.

It is another primary object of the present invention to improve the overall capability of ECG monitoring apparatus and to improve the reliability of data captured by such apparatus in a cost effective and convenient manner.

Therefore and according to a preferred aspect of the present invention, there is provided an ECG monitoring apparatus which is directly interconnected to a conventional printer, such as an HP ink jet or other desktop printer, such that ECG results can be printed onto plain paper without requiring a separate interconnection between a computer and the printer.

The ECG monitoring apparatus can be connected to the printer in the form of a snap-fitted, hinged or other form of releasable connection or alternatively can be more directly integrated therebetween. The monitoring apparatus preferably includes a display, as well as control means for controlling the operation of the printer once connected therewith, such that ECG results can be selectively displayed by the ECG monitoring apparatus and/or printed.

In another preferred embodiment, the monitoring apparatus, including the printer, can be mutually supported on a cart or other supporting structure wherein the printer and the monitoring apparatus can be integrated together or simply provided together on the same support (e.g., cart), thereby further enhancing overall capability and versatility of a monitoring system.

The herein described system can further provide for data entry using a keypad or other user interface that is provided with the monitoring portion of the assembly, permitting patient specific information to be added into a memory. For example, the monitoring system can be equipped with a programmable ASIC.

An advantage of the present invention is that color or other forms of plain paper printing is possible, thereby eliminating the need for thermal paper and associated problems resulting therefrom. Furthermore, the inventive concepts described herein are designed to be universally applicable to literally any form of commercially available printer without significant modifications being required.

These and other objects, features, and advantages will become apparent from the following Detailed Description which should be read in conjunction with the accompanying drawings.

### BRIEF DESCRIPTION OF THE DRAWINGS

FIG. 1 is a partial front perspective view of an ECG monitoring apparatus including a shroud section that is removably detached from a conventional printer and made in accordance with a first embodiment of the present invention;

FIG. 2 is a partial front perspective view of the ECG monitoring apparatus of FIG. 1 in an assembled configuration;

FIG. 3 is a partial front perspective view of an ECG monitoring apparatus made in accordance with a second embodiment of the present invention, herein illustrating the apparatus in an open position;

FIG. 4 is a front perspective view of the ECG monitoring apparatus of FIG. 3 in a closed operational configuration;

FIG. 5 is a front perspective view of an ECG monitoring apparatus in accordance with a third embodiment of the present invention;

FIG. 6 is a side view of an ECG monitoring apparatus made in accordance with a fourth embodiment of the present invention, illustrating the apparatus in a partially assembled condition;

FIG. 7 is a front perspective view of the ECG monitoring apparatus of FIG. 6, illustrating the apparatus in an assembled configuration;

FIG. 8 is a side view of an ECG monitoring apparatus made in accordance with a fifth embodiment of the present invention, illustrating the apparatus in a partially assembled condition;

FIG. 9 is a front perspective view of the ECG monitoring apparatus of FIG. 8, illustrating the apparatus in an assembled condition;

FIG. 10 is a top perspective view of an ECG monitoring apparatus made in accordance with a sixth embodiment of the present invention; and

FIG. 11 is a top perspective view of an ECG monitoring apparatus made in accordance with a seventh embodiment of the present invention.

### DETAILED DESCRIPTION

The following description relates to certain embodiments of an ECG monitoring apparatus and several adaptive means for combining same with a conventional computer ink-jet or other form of printer. It will be understood that variations and modifications are possible within the intended scope and spirit of the invention and that this description is intended only to provide suitable examples.

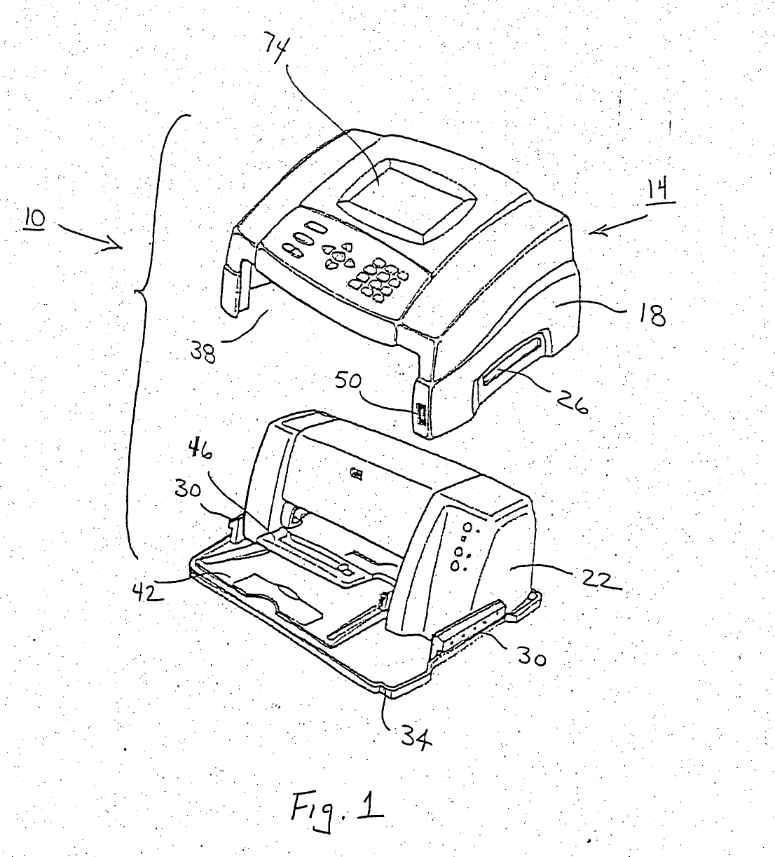

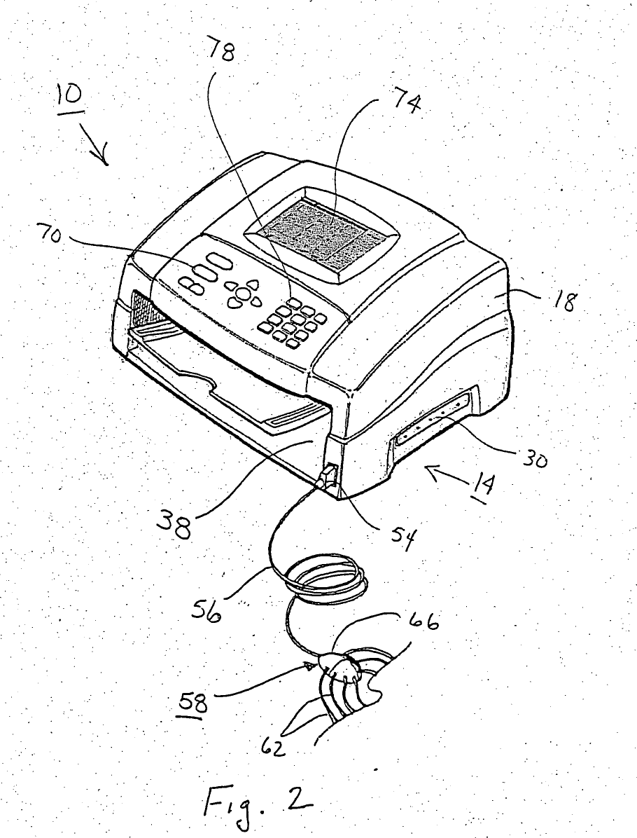

Referring to FIGS. 1 and 2, there is shown an apparatus made in accordance with a first embodiment of the present invention. The apparatus 10 includes a ECG monitoring portion 14 comprising a cover or shroud assembly 18 that is removably attachable to a conventional computer printer 22, such as any Hewlett Packard ink jet printer, such as those, for example, in the Laser Jet family of products. In this instance, the shroud assembly 18 includes a pair of shell-like members preferably constructed from a moldable plastic material, including a top piece 33 sized to substantially cover the entire periphery (top, sides, and rear) of the printer 22 and a base 34 disposed beneath the printer. The top piece 33 according to this embodiment is releasably snap-fitted through slots 26 that engage with corresponding tabs 30 projecting from lateral sides of the base 34, the tabs being in compressive contact with lateral sides of the printer 22.

The top piece 33 further includes a slotted frontal portion 38 that accommodates the paper dispense tray 42 and paper loader 46 of the retained printer 22 and provides user access therewith once the shroud assembly 18 is attached, as shown in FIG. 2. The shroud assembly 18 includes control circuitry therein and electrically interfaces with the printer 22 by means of cabling provided in the rear thereof that interfaces with the serial port of the printer. Alternately, other suitable connections can be employed to connect same.

The shroud assembly 18 as noted is part of an ECG monitoring portion 14 of the overall assembly 10 and includes a port 50 that is sized to receive the snap-fitting connector 54 of an electrode assembly 58. The electrode assembly 58 includes a number of patient connectable leads 62 having attached electrodes that are tethered individually and releasably connected to the outer periphery of a central housing 66, the housing being extremely compact and including therein a microprocessor or a digital signal processor (DSP) as well as an analog to digital converter for capturing signals from the electrodes and transmitting same to the shroud section 18 through a cable 56 terminating in the snap-fitting connector 54. The top piece 33 of the shroud assembly 18 further includes a user control interface 70 comprising a plurality of control buttons 70 that are used to operate the printer as well as an LCD 74 capable of producing a real-time graphical depiction of the waveforms produced by the multiple leads 62 (e.g., 12 lead, 15 lead) of the electrode assembly 58. Preferably, the assembly 10 can be battery-powered or can derive power from the printer 22, the electrode assembly 58, or a separate power supply.

As noted, the interconnection of the shroud assembly 18 of the ECG monitoring portion 14 to the printer 22 includes not only a mechanical connection but also a suitable electrical connection through either a USB, RS-232 or other suitable connection, enabling the ECG waveform signals produced by the electrode assembly 58 to be printed upon demand through use control buttons provided in the user interface 70. The user interface 70 further includes an alphanumeric keypad 78 wherein the shroud assembly 18 can further include a programmable ASIC so as to permit data entry, such as patient-related data, to be stored.

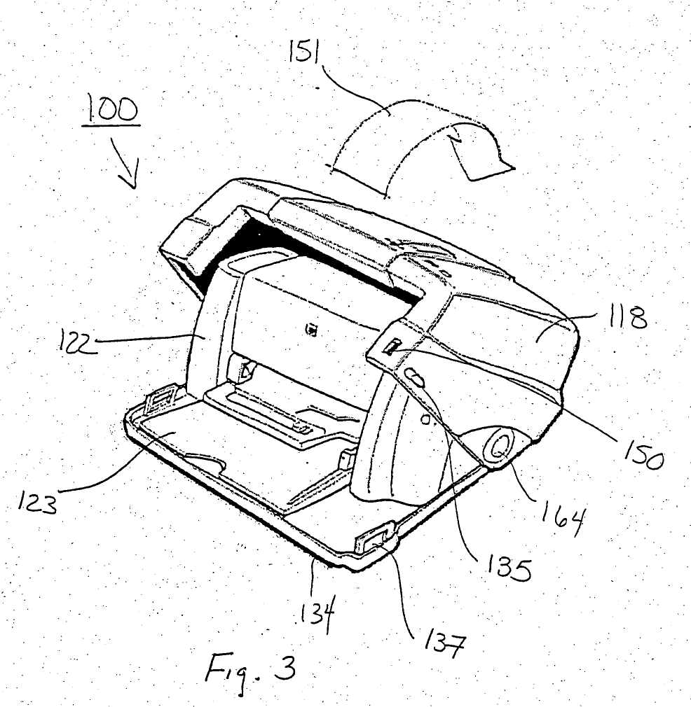

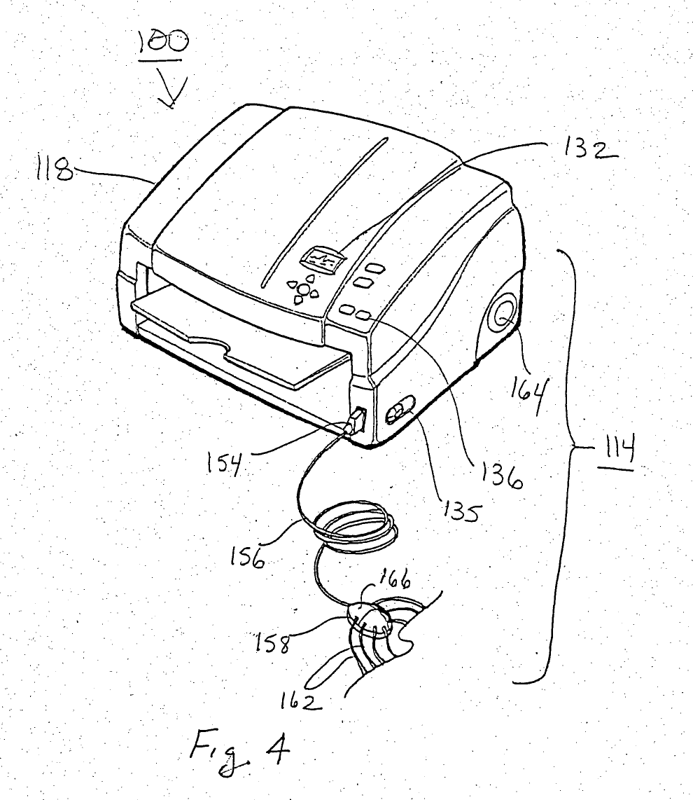

Variations upon this embodiment are herein described by way of example. Referring to FIGS. 3 and 4, an assembly 100 in accordance with a second embodiment is illustrated. The assembly 110 also includes an ECG monitoring section 114 is used in conjunction with a conventional printer 122, such as previously described. In lieu of a tab/slot connection, a shroud or cover assembly 118 includes a top piece 133 that is pivotally connected via a hinged connection 164 to the rear of a corresponding base 134, the printer 122 being retained therein, permitting the retained printer to be accessed as needed from the front side 123 thereof, for example, to remove paper, or to replace paper and/or toner cartridges. The top piece 133 includes a pair of sliders 135 on lateral sides thereof which engage corresponding slots 137 in the base 134 thereby permitting the top piece to be opened and closed, as shown by arrow 151 in FIG. 3.

It should be noted in passing that the shroud assembly 118 of FIGS. 3 and 4 is slightly different in appearance and functionality in terms of a provided LCD/display 132 and/or user control interface 136. This specific illustration is merely intended to be exemplary and therefore it should be readily apparent to one of sufficient skill that certain depicted features, for example, the necessity or size of a display can be easily varied or modified depending on needs and requirements of a given system or printer or user interface.

Otherwise, this version is similar to the preceding wherein an electrode assembly 158 is attached by means of a snap-fitting connector 154 at the end of a tethered cable 156, the electrode assembly including a central housing 166 and a set of releasably attached leads 168 to which electrodes are connected for attachment to the body of a patient. The central housing 166 preferably includes an analog to digital converter and a microprocessor for processing and filtering the signals from the appropriate leads. The snap-fitting connector 154 is fitted into a port 150 providing electrical connection with the shroud assembly 118 and in which prints can be made using buttons provided on the control user interface 136.

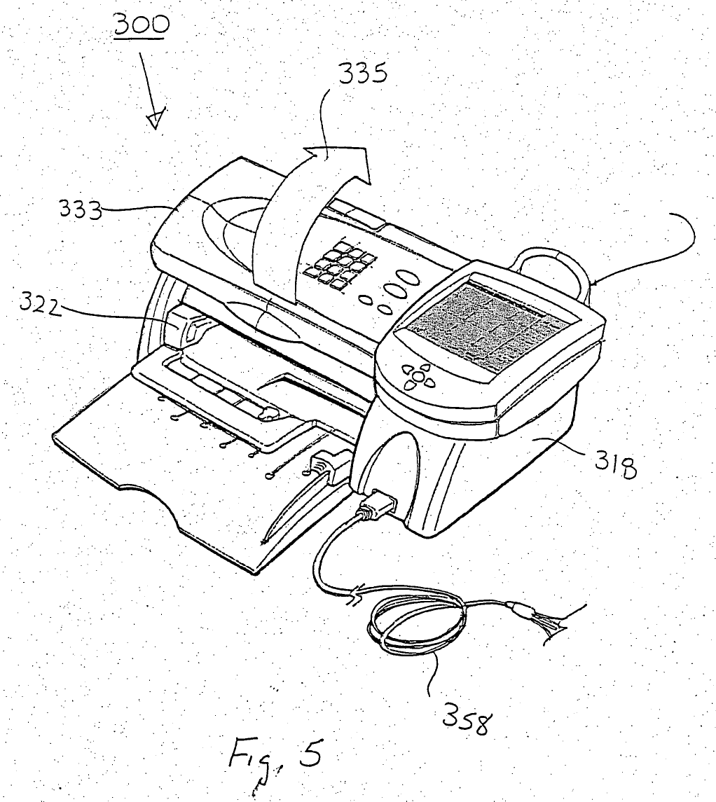

Still further variations are possible depending, for example, on the location of the serial port of the printer being utilized. According to FIG. 5, and as opposed to a cover-like assembly which essentially houses the printer, an ECG monitoring assembly 318 can be otherwise mounted or integrated with a conventional printer 322. In this embodiment, the ECG monitoring assembly 318 is attached to a printer 322, the monitoring assembly including a flip-up top piece 333 which permits access, see arrow 335, to the toner cartridges of the printer 322 once attached thereto through its serial port. The monitoring assembly 318, as in the preceding versions, includes control circuitry as well as a control user interface and an LCD and is tethered, also as in the preceding, to an ECG electrode assembly 358, partially shown in this Fig. A storage receptacle 370 is also included for storing spare leads and similar apparatus.

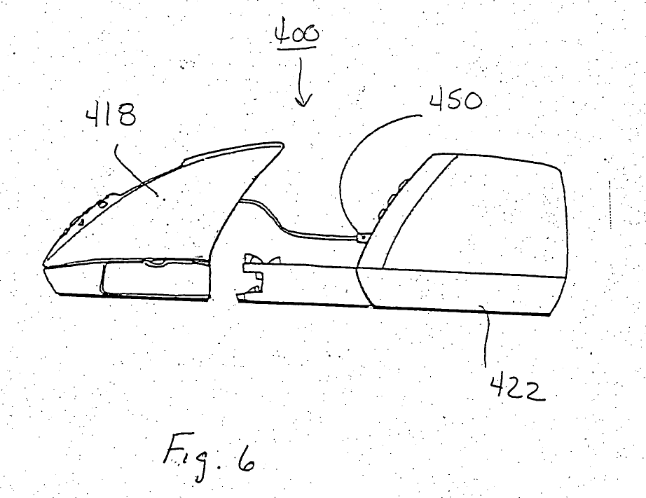

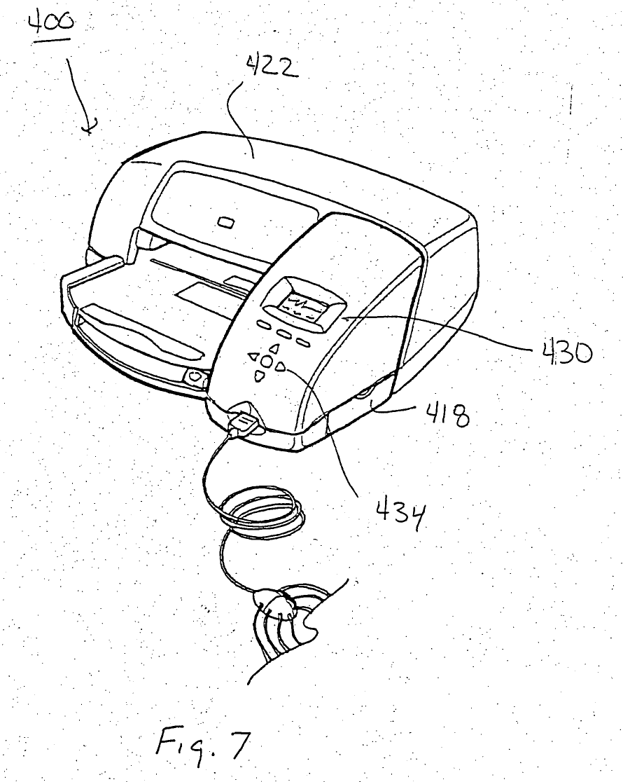

Still other variations are possible. For example and as shown in the assembly 400 depicted in FIGS. 6 and 7, a printer 422 is illustrated that includes a front port serial connection, shown more adequately in FIG. 6. In this instance, a separate releasably attached ECG monitoring assembly 418 is attached by means to the front of the printer 422, the assembly including an LCD 430 and a user interface 434 as well as control circuitry, as previously described, which permits operation of the printer using the interface once connected as shown in FIG. 6 using a USB or other suitable cable 450 extending from the rear of the monitoring assembly 418 that is then connected electrically to the serial port of the printer. The monitoring assembly 418 is relatively compact and is positioned over the existing user interface of the printer 422 so as not to interfere with access to the paper tray or toner cartridges.

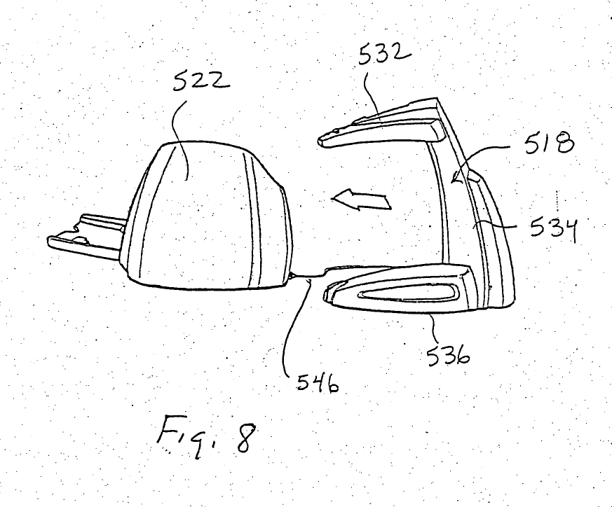

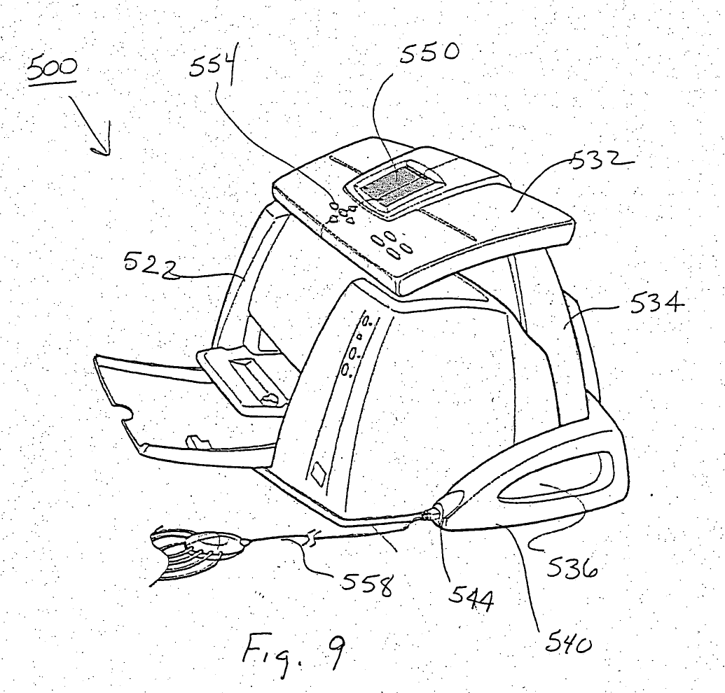

FIGS. 8 and 9 depict an assembly 500 made in accordance with a fifth embodiment of the present invention. In this example, the printer 522 includes a rear serial connection port. An ECG monitoring portion 518 of the assembly 500 is constructed with a top piece 532, a rear piece 534 and a base 536, respectively, so as to essentially support the top, bottom and rear, respectively, of a suitably positioned printer 522 as shown in FIG. 9. The base 536 includes a pair of arms 540, one of which includes a port 544 for receiving an ECG electrode assembly 558 such as previously described.

The top piece 532 of the monitoring portion 518 includes an LCD 550 as well as a user interface 554 for controlling the operation of the printer 522 once connected through a serial cord 546. In this instance, the printer 522 can also be operated manually using the printer controls which are also accessible.

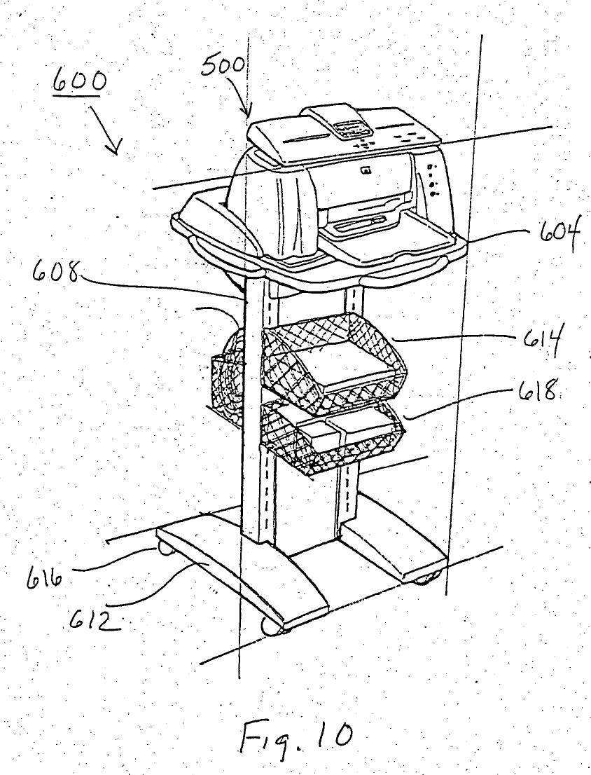

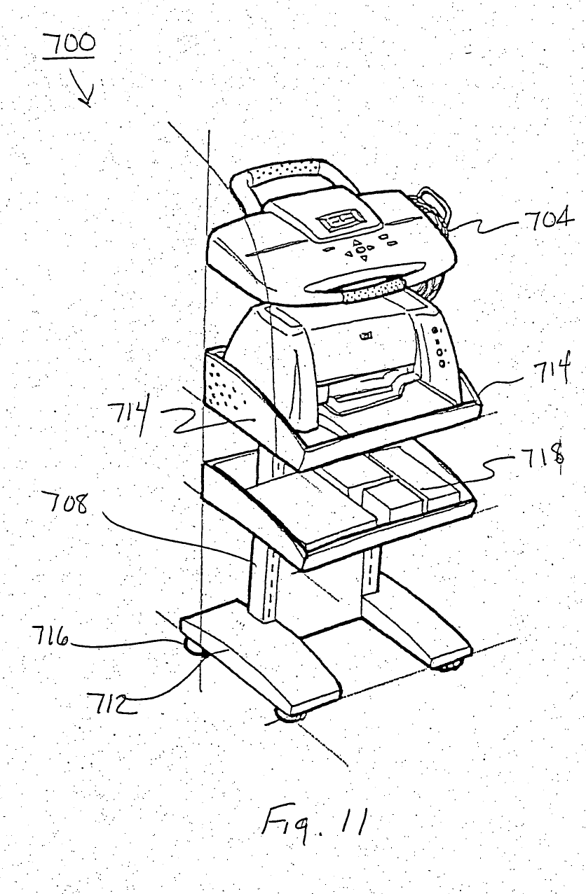

Rather than using a table or desk-top version as previously described in each of the foregoing, FIGS. 10 and 11 depict versions of the present invention whereby a conventional printer and ECG monitoring apparatus can be maintained within a single supporting structure. Referring to FIG. 10, the printer and ECG monitoring apparatus can be integrally incorporated, for example, by means of an assembly such as 500 previously described above in FIGS. 8 and 9, on the shelf of a wheeled cart 608. The cart 608 includes a vertical support and a plurality of shelves or as in the case of this embodiment, storage baskets 614, 618 to permit storage of items, such as spare leads, electrodes, and the like. The vertical support of the cart 608 is connected to a horizontal base 612, having wheels or casters 618, to permit wheeled movement of the structure.

An alternative design is illustrated in FIG. 11 in which a cart 708 includes a plurality of shelves 714, 718 attached to a vertical support, wherein a printer 722 and monitoring assembly 726 such as previously described are separately maintained thereupon. As in the preceding, the cart 708 includes a horizontal base 712, as well as wheels 716 or casters to permit movement of the assembly.

Carts or similar supporting structures as depicted herein provides advantages for the physician or caregiver in terms of overall mobility and adaptability. The cart further includes sufficient storage capability for spare leads, electrodes, gel, cables, paper and the like. Though the preceding are exemplary, it should be readily apparent that similar variations could easily be imagined by one of sufficient skill in the field.

### PARTS LIST FOR FIGS. 1-11

10 apparatus

14 ECG monitoring portion

18 cover or shroud assembly

22 printer

26 slots

30 tabs

33 top piece

34 base

38 slotted frontal portion

42 paper dispense tray

46 paper loader

50 port

54 snap-fitting connector

56 cable

58 electrode assembly

62 releasably attached leads

66 central housing

70 user control interface

74 LCD

78 keypad

100 assembly

114 ECG monitoring portion

118 shroud assembly

122 printer

123 front

132 LCD

133 top piece

134 base

135 sliders

136 user control interface

137 slots

150 port

151 arrow

154 snap-fitting connector

156 cable

158 electrode assembly

162 releasably connected leads

164 hinged connection

166 central housing

300 assembly

318 monitoring assembly

322 printer

333 top piece

335 arrow

358 electrode assembly

370 storage receptacle

400 assembly

418 monitoring assembly

422 printer

430 LCD

434 user interface

450 serial cable

500 assembly

518 ECG monitoring assembly

522 printer

532 top piece

534 back piece

536 base

540 arm

544 port

546 serial cable

550 LCD

554 user interface

558 electrode assembly

600 assembly

608 cart

612 base

614 storage basket

616 wheels

618 storage basket

700 assembly

704 shelf

708 cart

712 base

714 shelf

716 wheels

718 shelf

While the present invention has been particularly shown and described with reference to the preferred mode as illustrated in the drawing, it will be understood by one skilled in the art that various changes in detail may be effected therein without departing from the spirit and scope of the invention.

We claim:

1. An ECG monitoring apparatus comprising:
an electrode/lead assembly attachable to a patient;
a data acquisition module attached to said electrode/lead assembly; and
a printer directly connected to said data acquisition module.

2. An ECG monitoring apparatus as recited in claim 1, wherein said data acquisition module includes an A/D converter.

3. An ECG monitoring apparatus as recited in claim 1, wherein said printer is a conventional computer printer.

4. An ECG monitoring apparatus as recited in claim 1, wherein the data acquisition module is housed within the printer.

5. An ECG monitoring apparatus as recited in claim 1 wherein said printer is an ink-jet computer printer.

6. An ECG monitoring apparatus as recited in claim 1, including a supporting structure.

7. An ECG monitoring apparatus as recited in claim 6, wherein said supporting structure is a cart.

8. An ECG monitoring apparatus as recited in claim 7, wherein said cart includes at least one storage area for retaining loose items for storage thereof.

9. An ECG monitoring apparatus as recited in claim 8, wherein said at least one storage area is a drawer.

10. An ECG monitoring apparatus as recited in claim 9, wherein said at least one storage area is a shelf.

11. An ECG monitoring apparatus as recited in claim 1, including a user interface for operating at least one of said printer, said electrode/lead assembly and said data acquisition module.

12. An ECG monitoring apparatus as recited in claim 1, including a display for displaying data acquired by said data acquisition module.

13. An ECG monitoring apparatus as recited in claim 1, wherein said data acquisition module is a compact housing commonly retaining a plurality of leads from said electrode/lead assembly, said data acquisition module connected to said printer.

14. An ECG monitoring apparatus as recited in claim 13, wherein said data acquisition module is wirelessly connected to said printer, said module including means for bi-directionally communicating between said printer.

15. An ECG monitoring apparatus as recited in claim 4, wherein the ends of a plurality of leads from said electrode/lead assembly are connected to said data acquisition module.

16. An ECG monitoring apparatus as recited in claim 1, wherein said data acquisition module and said electrode/lead assembly are removably attachable to said printer.

17. An ECG monitoring apparatus as recited in claim 1, further including a shroud assembly of receiving connections from said electrode/lead assembly and said data acquisition module said shroud assembly being configured to interconnect a conventional table to computer printer therewith.

18. An ECG monitoring apparatus as recited in claim 17, wherein said shroud assembly includes a shroud housing that covers at least a portion of said printer and includes a user interface for operating said printer and said ECG monitoring apparatus.

19. An ECG monitoring apparatus as recited in claim 18, wherein said shroud assembly includes a display.

20. An ECG monitoring apparatus as recited in claim 18, wherein said shroud assembly includes a programmable ASIC.

21. An ECG monitoring apparatus as recited in claim 18, wherein said data acquisition module is a compact housing commonly retaining the ends of leads from said electrode/lead assembly, said data acquisition module being tethered to said shroud assembly.
